(19) United States
(12) Patent Application Publication
(10) Pub. No.: US 2008/0300138 A1
(43) Pub. Date: Dec. 4, 2008

(54) Dioxazinyl-Substituted Thienylsulphonylaminocarbonyl Compounds

(76) Inventors: Ernst R. F. Gesing, Erkrath-Hochdahl (DE); Dieter Feucht, Eschborn (DE); Heinz Kehne, Hofheim (DE); Thomas Auler, Leichlingen (DE); Martin Hills, Idstein (DE)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/658,537

(22) PCT Filed: Jul. 13, 2005
(86) PCT No.: PCT/EP05/07580

§ 371 Date: Jan. 25, 2007

(30) Foreign Application Priority Data
Jul. 28, 2004 (DE) 10 2004 036 551.2

(51) Int. Cl. A01N 43/88; C07D 413/14

(52) U.S. Cl. 504/223; 544/65

(57) ABSTRACT

The invention relates to novel substituted thienylsulfonylaminocarbonyl compounds of the formula (I),

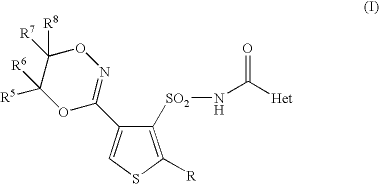

in which R, Het and R⁵ to R⁸ are as defined in the description,
to processes and intermediates for their preparation and to their use as herbicides.

The invention relates to novel substituted thienylsulfonylaminocarbonyl compounds, to processes and intermediates for their preparation and to their use as herbicides.

It is already known that certain dioxazinyl-substituted thienylsulfonylaminocarbonyl compounds have herbicidal properties (cf. U.S. Pat. No. 5,476,936). However, the herbicidal activity of these known compounds is not entirely satisfactory.

This invention now provides the novel substituted thienylsulfonylaminocarbonyl compounds of the formula (I)

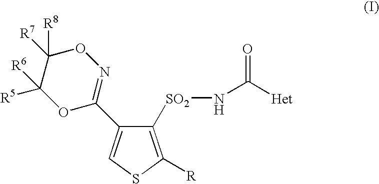

in which
Het is

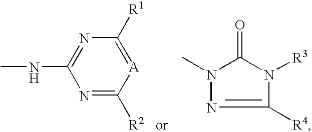

A is nitrogen or a CH grouping,
R is hydrogen, cyano, nitro, halogen, is in each case optionally cyano-, halogen- or C₁-C₄-alkoxy-substituted alkyl, alkoxy, alkoxycarbonyl, alkylthio, alkylsulfinyl or alkylsulfonyl having in each case 1 to 6 carbon atoms in the alkyl group, or is in each case optionally cyano- or halogen-substituted alkenyl, alkynyl, alkenyloxy or alkynyloxy having in each case 2 to 6 carbon atoms in the alkenyl or alkynyl group,
R¹ is hydrogen, is halogen, is in each case optionally cyano-, halogen- or C₁-C₄-alkoxy-substituted alkyl, alkoxy, alkylthio, alkylamino or dialkylamino having in each case 1 to 4 carbon atoms in the alkyl groups, or is in each case optionally cyano-, halogen-, C₁-C₄-alkyl- or C₁-C₄-alkoxy-substituted phenoxy, oxetanyloxy, furyloxy or tetrahydrofuryloxy,
R² is hydrogen, is halogen, is in each case optionally cyano-, halogen- or C₁-C₄-alkoxy-substituted alkyl, alkoxy, alkylthio, alkylamino or dialkylamino having in each case 1 to 4 carbon atoms in the alkyl groups, or is in each case optionally cyano-, halogen-, C₁-C₄-alkyl- or C₁-C₄-alkoxy-substituted phenoxy, oxetanyloxy, furyloxy or tetrahydrofuryloxy,
R³ is hydrogen, hydroxyl, amino, cyano, is C₂-C₁₀-alkylideneamino, is optionally fluorine-, chlorine-, bromine-, cyano-, C₁-C₄-alkoxy-, C₁-C₄-alkyl-carbonyl- or C₁-C₄-alkoxy-carbonyl-substituted alkyl having 1 to 6 carbon atoms, is in each case optionally fluorine-, chlorine- and/or bromine-substituted alkenyl or alkynyl having in each case 2 to 6 carbon atoms, is in each case optionally fluorine-, chlorine-, bromine-, cyano-, C₁-C₄-alkoxy- or C₁-C₄-alkoxy-carbonyl-substituted alkoxy, alkylamino or alkylcarbonylamino having in each case 1 to 6 carbon atoms in the alkyl group, is alkenyloxy having 3 to 6 carbon atoms, is dialkylamino having in each case 1 to 4 carbon atoms in the alkyl groups, is in each case optionally fluorine-, chlorine-, bromine-, cyano- and/or C₁-C₄-alkyl-substituted cycloalkyl, cycloalkylamino or cycloalkylalkyl having in each case 3 to 6 carbon atoms in the alkyl group and, if appropriate, 1 to 4 carbon atoms in the alkyl moiety, or is in each case optionally fluorine-, chlorine-, bromine-, cyano-, nitro-, C₁-C₄-alkyl-, trifluoromethyl- and/or C₁-C₄-alkoxy-substituted aryl or arylalkyl having in each case 6 or 10 carbon atoms in the aryl group and, if appropriate, 1 to 4 carbon atoms in the alkyl moiety.
R⁴ is hydrogen, hydroxyl, mercapto, amino, cyano, fluorine, chlorine, bromine, iodine, is optionally fluorine-, chlorine-, bromine-, cyano-, C₁-C₄-alkoxy-, C₁-C₄-alkyl-carbonyl- or C₁-C₄-alkoxy-carbonyl-substituted alkyl having 1 to 6 carbon atoms, is in each case optionally fluorine-, chlorine- and/or bromine-substituted alkenyl or alkynyl having in each case 2 to 6 carbon atoms, is in each case optionally fluorine-, chlorine-, cyano-, C₁-C₄-alkoxy- or C₁-C₄-alkoxy-carbonyl-substituted alkoxy, alkylthio, alkylamino or alkylcarbonylamino having in each case 1 to 6 carbon atoms in the alkyl group, is alkenyloxy, alkynyloxy, alkenylthio, alkynylthio, alkenylamino or alkynylamino having in each case 3 to 6 carbon atoms in the alkenyl or alkynyl group, is dialkylamino having in each case 1 to 4 carbon atoms in the alkyl groups, is in each case optionally fluorine-, chlorine-, bromine-, cyano- and/or C₁-C₄-alkyl-substituted cycloalkyl, cycloalkenyl, cycloalkyloxy, cycloalkylthio, cycloalkylamino, cycloalkylalkyl, cycloalkylalkoxy, cycloalkylalkylthio or cycloalkylalkylamino having in each case 3 to 6 carbon atoms in the cycloalkyl or cycloalkenyl group and, if appropriate, 1 to 4 carbon atoms in the alkyl moiety, or is in each case optionally fluorine-, chlorine-, bromine-, cyano-, nitro-, C₁-C₄-alkyl-, trifluoromethyl-, C₁-C₄-alkoxy- and/or C₁-C₄-alkoxy-carbonyl-substituted aryl, arylalkyl, aryloxy, arylalkoxy, arylthio, arylalkylthio, arylamino or arylalkylamino having in each case 6 or 10 carbon atoms in the aryl group and, if appropriate, 1 to 4 carbon atoms in the alkyl moiety, and
R⁵, R⁶, R⁷ and R⁸ independently of one another are hydrogen, halogen, cyano or thiocyanato or are in each case optionally halogen-substituted alkyl, alkoxy, alkylthio, alkylsulfinyl, alkylsulfonyl, alkylamino, alkylcarbonyl, alkoxycarbonyl or alkylaminocarbonyl having in each case 1 to 3 carbon atoms in the alkyl moiety,
   and salts of compounds of the formula (I).

Saturated or unsaturated hydrocarbon groupings, such as alkyl, alkenyl or alkynyl, are in each case straight-chain or branched as far as this is possible—including in combination with heteroatoms, such as in alkoxy.

Optionally substituted radicals may be mono- or polysubstituted, where in the case of polysubstitution the substituents can be identical or different.
R is preferably hydrogen, cyano, fluorine, chlorine, bromine, is preferably in each case optionally cyano-, fluorine-, chlorine-, methoxy- or ethoxy-substituted methyl, ethyl, n- or i-propyl, n-, i-, s- or t-butyl, methoxy, ethoxy, n- or i-propoxy, methoxycarbonyl, ethoxycarbonyl, n- or i-propoxycarbonyl, methylthio, ethylthio, n- or i-propylthio, methylsulfinyl, ethylsulfinyl, methylsulfonyl or ethylsulfonyl, or is preferably in each case optionally cyano-, fluorine- or chlorine-substituted propenyl, butenyl, propynyl, butynyl, propenyloxy, butenyloxy, propynyloxy or butynyloxy.
R is particularly preferably fluorine, chlorine, bromine, is particularly preferably in each case optionally cyano-, fluorine-, chlorine-, methoxy- or ethoxy-substituted methyl, ethyl, n- or i-propyl, n-, i-, s- or t-butyl, methoxy, ethoxy, n- or i-propoxy.
R is most preferably methyl, ethyl, n- or i-propyl.
R¹ is preferably hydrogen, fluorine, chlorine, bromine, iodine, or is preferably in each case optionally cyano-, fluorine-, chlorine-, methoxy- or ethoxy-substituted methyl, ethyl, n- or i-propyl, methoxy, ethoxy, n- or i-propoxy, methylthio, ethylthio, n- or i-propylthio, methylamino, ethylamino, n- or i-propylamino, dimethylamino or diethylamino.
R¹ is particularly preferably fluorine, chlorine, bromine, or iodine, or is particularly preferably optionally fluorine- or chlorine-substituted methyl, ethyl, n- or i-propyl, methoxy, ethoxy, n- or i-propoxy or is particularly preferably dimethylamino.
R² is preferably fluorine, chlorine, bromine, iodine, or is preferably in each case optionally cyano-, fluorine-, chlorine-, methoxy- or ethoxy-substituted methyl, ethyl, n- or i-propyl, methoxy, ethoxy, n- or i-propoxy, methylthio, ethylthio, n- or i-propylthio, methylamino, ethylamino, n- or i-propylamino, dimethylamino or diethylamino.
R² is particularly preferably fluorine, chlorine, bromine, or iodine, or is particularly preferably optionally fluorine- or chlorine-substituted methyl, ethyl, n- or i-propyl, methoxy, ethoxy, n- or i-propoxy or is particularly preferably dimethylamino.
R³ is preferably hydrogen, hydroxyl, amino, is preferably in each case optionally fluorine-, chlorine-, cyano-, methoxy- or ethoxy-substituted methyl, ethyl, n- or i-propyl, n-, i-, s- or t-butyl, is preferably in each case optionally fluorine-, chlorine- and/or bromine-substituted ethenyl, propenyl, butenyl, propynyl or butynyl, is preferably in each case optionally fluorine-, chlorine-, cyano-, methoxy- or ethoxy-substituted methoxy, ethoxy, n- or i-propoxy, n-, i-, s- or t-butoxy, methylamino, ethylamino, n- or i-propylamino, n-, i-, s- or t-butylamino, is preferably propenyloxy or butenyloxy, is preferably dimethylamino or diethylamino, is preferably in each case optionally fluorine-, chlorine-, methyl- and/or ethyl-substituted cyclopropyl, cyclobutyl, cyclopentyl, cyclohexyl, cyclopropylamino, cyclobutylamino, cyclopentylamino, cyclohexylamino, cyclopropylmethyl, cyclobutylmethyl, cyclopentylmethyl or cyclohexylmethyl, or is preferably in each case optionally fluorine-, chlorine-, methyl-, trifluoromethyl- and/or methoxy-substituted phenyl or benzyl.
R³ is particularly preferably methyl, ethyl, n- or i-propyl or cyclopropyl.
R⁴ is preferably hydrogen, hydroxyl, mercapto, amino, cyano, fluorine, chlorine, bromine, is preferably in each case optionally fluorine-, chlorine-, cyano-, methoxy-, ethoxy-, n- or i-propoxy-, acetyl-, propionyl-, n- or i-butyroyl-, methoxycarbonyl-, ethoxycarbonyl-, n- or i-propoxycarbonyl-substituted methyl, ethyl, n- or i-propyl, n-, i-, s- or t-butyl, is preferably in each case optionally fluorine-, chlorine- and/or bromine-substituted ethenyl, propenyl, butenyl, ethynyl, propynyl or butynyl, is preferably in each case optionally fluorine-, chlorine-, cyano-, methoxy-, ethoxy-, n- or i-propoxy-, methoxycarbonyl-, ethoxycarbonyl-, n- or i-propoxycarbonyl-substituted methoxy, ethoxy, n- or i-propoxy, n-, i-, s- or t-butoxy, methylthio, ethylthio, n- or i-propylthio, n-, i-, s- or t-butylthio, methylamino, ethylamino, n- or i-propylamino, n-, i-, s- or t-butylamino, acetylamino or propionylamino, is preferably propenyloxy, butenyloxy, ethynyloxy, propynyloxy, butynyloxy, propenylthio, butenylthio, propynylthio, butynylthio, propenylamino, butenylamino, propynylamino or butynylamino, is preferably dimethylamino, diethylamino or dipropylamino, is preferably in each case optionally fluorine-, chlorine-, methyl- and/or ethyl-substituted cyclopropyl, cyclobutyl, cyclopentyl, cyclohexyl, cyclopentenyl, cyclohexenyl, cyclopropyloxy, cyclobutyloxy, cyclopentyloxy, cyclohexyloxy, cyclopropylthio, cyclobutylthio, cyclopentylthio, cyclohexylthio, cyclopropylamino, cyclobutylamino, cyclopentylamino, cyclohexylamino, cyclopropylmethyl, cyclobutylmethyl, cyclopentylmethyl, cyclohexylmethyl, cyclopropylmethoxy, cyclobutylmethoxy, cyclopentyl-methoxy, cyclohexylmethoxy, cyclopropylmethylthio, cyclobutylmethylthio, cyclopentylmethylthio, cyclohexylmethylthio, cyclopropylmethylamino, cyclobutylmethylamino, cyclopentylmethylamino or cyclohexylmethylamino, or is preferably in each case optionally fluorine-, chlorine-, bromine-, methyl-, trifluoromethyl-, methoxy- or methoxy-carbonyl-substituted phenyl, benzyl, phenoxy, benzyloxy, phenylthio, benzylthio, phenylamino or benzylamino.
R⁴ is particularly preferably methoxy, ethoxy, n- or i-propoxy, n-, i-, s- or t-butoxy.
R⁵, R⁶, R⁷ and R⁸ are preferably independently of one another hydrogen or methyl.
R⁵, R⁶, R⁷ and R⁸ are particularly preferably hydrogen.

The invention preferably also provides the sodium, potassium, magnesium, calcium, ammonium, C₁-C₄-alkylammonium, di-(C₁-C₄-alkyl)-ammonium-, tri-(C₁-C₄-alkyl)-ammonium, tetra-(C₁-C₄-alkyl)-ammonium, tri-(C₁-C₄-alkyl)-sulfonium, C₅- or C₆-cycloalkylammonium- and di-(C₁-C₂-alkyl)-benzylammonium salts of compounds of the formula (I), in which A, Het, R¹, R², R³, R⁴, R⁵, R⁶, R⁷ and R⁸ have the meaning given above as being preferred.

The general or preferred radical definitions given above apply both to the end products of the formula (I) and, correspondingly, to the starting materials and intermediates required in each case for the preparation. These radical definitions, can be combined with one another as desired, i.e. including combinations between the given ranges of preferred compounds.

Preference according to the invention is given to those compounds of the formula (I) which contain a combination of the meanings given above as being preferred.

Particular preference according to the invention is given to those compounds of the formula (I) which contain a combination of the meanings given above as being particularly preferred.

The novel substituted thienylsulfonylaminocarbonyl compounds of the formula (I) have strong herbicidal activity.

The novel substituted thienylsulfonylaminocarbonyl compounds of the formula (I) are obtained when

(a) substituted aminoazines of the formula (II)

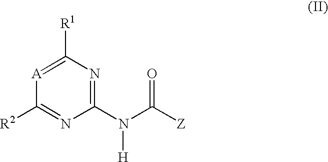

in which
A, R¹ and R² are as defined above and
Z is halogen, alkoxy or aryloxy
are reacted with thiophene derivatives of the formula (III)

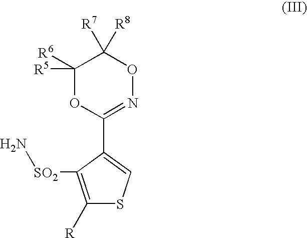

in which
R and R⁵ to R⁸ are as defined above,
if appropriate in the presence of a reaction auxiliary and if appropriate in the presence of a diluent,
or (b) substituted thiophene-3-sulfonamides of the formula (III)

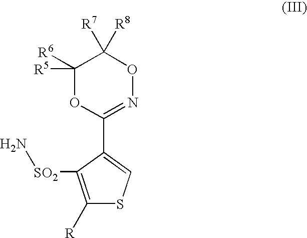

in which R and R⁵ to R⁸ are as defined above are reacted with substituted triazolinones of the formula (IV)

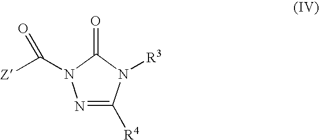

in which
R³ and R⁴ are as defined above and
Z′ is halogen, alkoxy, aryloxy or arylalkoxy,
if appropriate in the presence of a reaction auxiliary and if appropriate in the presence of a diluent,
and, if appropriate, the compounds of the formula (I) obtained by process (a) or (b) are converted by customary methods into salts.

Using, for example, 2-methoxycarbonylamino-4-methoxy-6-trifluoromethyl-1,3,5-triazine and 2-ethyl-4-(5,6-dihydro-[1,4,2]-dioxazin-3-yl)-thiophene-3-sulfonamide as starting materials, the course of the reaction in the process (a) according to the invention can be illustrated by the formula scheme given below:

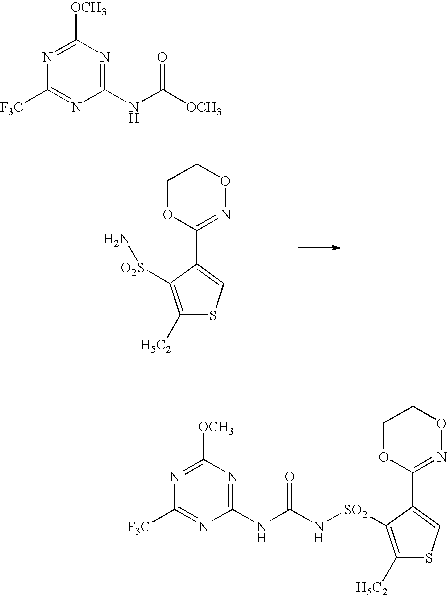

The formula (II) provides a general definition of the substituted aminoazines to be used as starting materials in the process according to the invention for preparing the compounds of the formula (I). In the formula (II), A, R¹ and R² preferably or in particular have those meanings which have already been given above in connection with the description of the compounds of the formula (I) as being preferred or as being particularly preferred for A, R¹ and R²; Z is preferably fluorine, chlorine, bromine, C₁-C₄-alkoxy or phenoxy, in particular chlorine, methoxy, ethoxy or phenoxy.

The starting materials of the formula (II) are known and/or can be prepared by processes known per se (cf. U.S. Pat. No. 4,690,707, DE 19501174).

The formula (III) provides a general definition of the thiophene derivatives further to be used as starting materials in the process according to the invention. In the formula (III), R and R⁵ to R⁸ preferably or in particular have those meanings which have already been given above in connection with the description of the compounds of the formula (I) as being preferred or as being particularly preferred for R and R⁵ to R⁸.

The substituted thiophene-3-sulfonamides of the formula (III) have hitherto not been disclosed in the literature; as such, they also form part of the subject-matter of the present invention. From among the compounds of the formula (III), preference is given to those in which R is not hydrogen.

The substituted thiophene-3-sulfonamides of the formula (III) are obtained when substituted thiophene-3-sulfonamides of the formula (V)

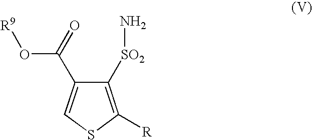

in which
R is as defined above and R⁹ is C₁-C₄-alkyl
are reacted with hydroxylamine hydrochloride and a substituted alkane of the formula (VI)

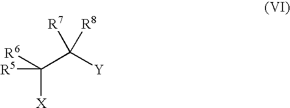

in which R⁵ to R⁸ are as defined above and
X and Y independently of one another are halogen, C₁-C₆-alkylcarbonyloxy, C₆-C₁₂-arylcarbonyloxy, C₁-C₆-alkylsulfonyloxy or C₁-C₆-arylsulfonyloxy,
if appropriate in the presence of a diluent and if appropriate in the presence of a reaction auxiliary (cf. the preparation example). Analogous processes are also known from U.S. Pat. No. 5,476,936 (cf. columns 11/12).

The starting materials of the formula (V) are known and/or can be prepared by processes known per se (cf. WO 01/05788, WO 01/10863).

The alkanes of the formula (VI) are commercially available as chemicals for synthesis or can be prepared by processes known per se (cf. U.S. Pat. No. 5,476,936, column 13).

Using, for example, 2-methyl-4-(5,6-dihydro-[1,4,2]-dioxazin-3-yl)-thiophene-3-sulfonamide and 4,5-dimethoxy-2-phenoxycarbonyl-2,4-dihydro-3H-1,2,4-triazol-3-one as starting materials, the course of the reaction in the process (b) according to the invention can be illustrated by the formula scheme below:

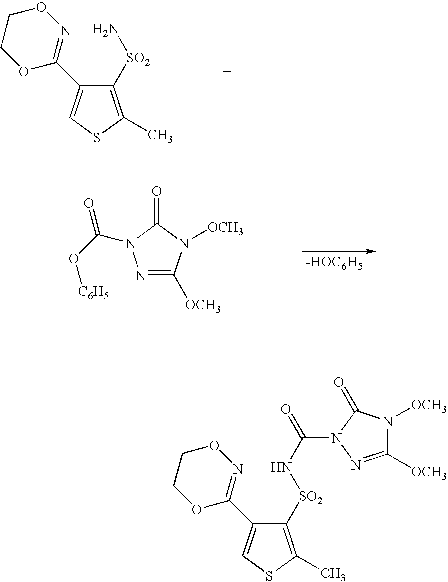

The formula (III) provides a general definition of the substituted thiophene-3-sulfonamides to be used as starting materials in the process (b) according to the invention for preparing compounds of the formula (I). They are the same compounds of the formula (III) used according to process (a) according to the invention.

The formula (IV) provides a general definition of the substituted triazolinones further to be used as starting materials in the process (b) according to the invention for preparing compounds of the formula (I). In the formula (IV), R³ and R⁴ preferably or in particular have those meanings which have already been given above in connection with the description of the compounds of the formula (I) according to the invention as being preferred or as being particularly preferred for R³ and R³.

The starting materials of the formula (IV) are known and/or can be prepared by processes known per se (cf. also notes in WO 01/05788). Z′ in the formula (IV) is preferably chlorine, bromine, methoxy, ethoxy, phenoxy or benzyloxy.

Suitable diluents for carrying out the processes (a) and (b) according to the invention and the process for preparing the intermediates of the formula (III) are especially inert organic solvents. These include, in particular, aliphatic, alicyclic or aromatic, optionally halogenated hydrocarbons, such as, for example, petrol, benzene, toluene, xylene, chlorobenzene, dichlorobenzene, petroleum ether, hexane, cyclohexane, dichloromethane, chloroform, carbon tetrachloride; ethers, such as diethyl ether, diisopropyl ether, dioxane, tetrahydrofuran or ethylene glycol dimethyl ether or ethylene glycol diethyl ether; ketones, such as acetone, butanone or methyl isobutyl ketone; nitriles, such as acetonitrile, propionitrile or benzonitrile; amides, such as N,N-dimethylformamide, N,N-dimethylacetamide, N-methylformanilide, N-methylpyrrolidone or hexamethylphosphoric triamide; esters, such as methyl acetate or ethyl acetate; and also sulfoxides, such as dimethyl sulfoxide.

The processes (a) and (b) according to the invention and the process for preparing the intermediates of the formula (III) are preferably carried out in the presence of a suitable reaction auxiliary. Suitable reaction auxiliaries are all customary inorganic or organic bases. These include, for example, alkaline earth metal or alkali metal hydrides, hydroxides, amides, alkoxides, acetates, carbonates or bicarbonates, such as, for example sodium hydride, sodium amide, sodium methoxide, sodium ethoxide, potassium tert-butoxide, sodium hydroxide, potassium hydroxide, ammonium hydroxide, sodium acetate, potassium acetate, calcium acetate, ammonium acetate, sodium carbonate, potassium carbonate, potassium bicarbonate, sodium bicarbonate or ammonium carbonate, and also tertiary amines, such as trimethylamine, triethylamine, tributylamine, N,N-dimethylaniline, pyridine, N-methylpiperidine, N,N-dimethylaminopyridine, diazabicyclooctane (DABCO), diazabicyclononene (DBN) or diazabicycloundecene (DBU).

When carrying out the process (a) according to the invention, the reaction temperatures can be varied within a relatively large range. In general, the process is carried out at temperatures between −20° C. and +150° C., preferably between −10° C. and +120° C.

When carrying out the process (b) according to the invention, the reaction temperatures can be varied within a relatively wide range. In general, the process is carried out at temperatures between −20° C. and +150° C., preferably between 0° C. and +100° C.

When carrying out the process according to the invention for preparing the intermediates of the formula (III), the reaction temperatures can be varied within a relatively wide range. In general, the process is carried out at temperatures between 0° C. and +150° C., preferably between 10° C. and +80° C.

The processes (a) and (b) according to the invention and the process for preparing intermediates of the formula (III) are generally carried out under atmospheric pressure. However, it is also possible to carry out the processes according to the invention under elevated or reduced pressure—in general between 0.1 bar and 10 bar.

For carrying out the processes (a) and (b) according to the invention and the process for preparing intermediates of the formula (III), the starting materials are generally employed in approximately equimolar amounts. However, it is also possible to use a relatively large excess of one of the components. The reaction is generally carried out in a suitable diluent in the presence of a reaction auxiliary, and the reaction mixture is generally stirred at the required temperature for a number of hours. Work-up is carried out by customary methods (cf. the preparation examples).

If appropriate, salts may be prepared from the compounds of the formula (I) according to the invention. Such salts are obtained in a simple manner by customary methods for forming salts, for example by dissolving or dispersing a compound of the formula (I) in a suitable solvent, such as, for example, methylene chloride, acetone, tert-butyl methyl ether or toluene, and adding a suitable base. The salts can then be isolated—if appropriate after prolonged stirring—by concentration or filtration with suction.

The active compounds according to the invention can be used as defoliants, desiccants, haulm killers and, especially, as weedkillers. Weeds in the broadest sense are understood to mean all plants which grow in locations where they are undesired. Whether the substances according to the invention act as total or selective herbicides depends essentially on the amount used.

The active compounds according to the invention can be used, for example, in connection with the following plants:

Dicotyledonous weeds of the genera: Abutilon, Amaranthus, Ambrosia, Anoda, Anthemis, Aphanes, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium. Dicotyledonous crops of the genera: Arachis, Beta, Brassica, Cucumis, Cucurbita, Helianthus, Daucus, Glycine, Gossypium, Ipomoea, Lactuca, Linum, Lycopersicon, Nicotiana, Phaseolus, Pisum, Solanum, Vicia.

Monocotyledonous weeds of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Monocotyledonous crops of the genera: Allium, Ananas, Asparagus, Avena, Hordeum, Oryza, Panicum, Saccharum, Secale, Sorghum, Triticale, Triticum, Zea.

However, the use of the active compounds according to the invention is in no way restricted to these genera, but also extends in the same manner to other plants.

The active compounds according to the invention are suitable, depending on the concentration, for the total control of weeds, for example on industrial terrain and rail tracks, and on paths and areas with and without tree plantings. Similarly, the active compounds according to the invention can be employed for controlling weeds in perennial crops, for example forests, decorative tree plantings, orchards, vineyards, citrus groves, nut orchards, banana plantations, coffee plantations, tea plantations, rubber plantations, oil palm plantations, cocoa plantations, soft fruit plantings and hop fields, on lawns, turf and pastureland, and for the selective control of weeds in annual crops.

The compounds according to the invention have strong herbicidal activity and a broad activity spectrum when used on the soil and on above-ground parts of plants.

To a certain extent they are also suitable for the selective control of monocotyledonous and dicotyledonous weeds in monocotyledonous and dicotyledonous crops, both by the pre-emergence and by the post-emergence method.

At certain concentrations or application rates, the active compounds according to the invention can also be employed for controlling animal pests and fungal or bacterial plant diseases. If appropriate, they can also be used as intermediates or precursors for the synthesis of other active compounds.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and recombinant methods or by combinations of these methods, including the transgenic plants and including the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offsets and seeds.

Treatment according to the invention of the plants and plant parts with the active compounds is carried out directly or by allowing the compounds to act on their surroundings, environment or storage space by the customary treatment methods, for example by immersion, spraying, evaporation, fogging, scattering, painting on and, in the case of propagation material, in particular in the case of seeds, also by applying one or more coats.

The active compounds can be converted into the customary formulations such as solutions, emulsions, wettable powders, suspensions, powders, dusts, pastes, soluble powders, granules, suspoemulsion concentrates, natural and synthetic materials impregnated with active compound, and microencapsulations in polymeric materials.

These formulations are produced in a known manner, for example by mixing the active compounds with extenders, that is liquid solvents and/or solid carriers, optionally with the use of surfactants, that is emulsifiers and/or dispersants, and/or foam formers.

If the extender used is water, it is also possible to employ, for example, organic solvents as auxiliary solvents. Essentially, suitable liquid solvents are: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulfoxide, or else water.

Suitable solid carriers are: for example ammonium salts and ground natural minerals such as kaolins, clays, talc, chalk, quartz, attapulgite, montmorillonite or diatomaceous earth, and ground synthetic minerals such as finely divided silica, alumina and silicates; suitable solid carriers for granules are: for example crushed and fractionated natural rocks such as calcite, marble, pumice, sepiolite and dolomite, or else synthetic granules of inorganic and organic meals, and granules of organic material such as sawdust, coconut shells, corn cobs and tobacco stalks; suitable emulsifiers and/or foam formers are: for example nonionic and anionic emulsifiers such as polyoxyethylene fatty acid esters, polyoxyethylene fatty alcohol ethers, for example alkylaryl polyglycol ethers, alkylsulfonates, alkyl sulfates, arylsulfonates, or else protein hydrolysates; suitable dispersants are: for example lignosulfite waste liquors and methylcellulose.

Tackifiers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic colorants such as alizarin colorants, azo colorants and metal phthalocyanine colorants, and trace nutrients such as salts of iron, manganese, boron, copper, cobalt, molybdenum and zinc.

The formulations generally comprise between 0.1 and 95% by weight of active compound, preferably between 0.5 and 90%.

For controlling weeds, the active compounds according to the invention, as such or in their formulations, can also be used as mixtures with other agrochemically active compounds, for example known herbicides and/or substances which improve the compatibility with crop plants (“safeners”), finished formulations or tank mixes being possible. Also possible are thus mixtures with weedkillers comprising one or more known herbicides and a safener.

Possible components for the mixtures are known herbicides, for example acetochlor, acifluorfen (-sodium), aclonifen, alachlor, alloxydim (-sodium), ametryne, amicarbazone, amidochlor, amidosulfuron, anilofos, asulam, atrazine, azafenidin, azimsulfuron, beflubutamid, benazolin (-ethyl), benfuresate, bensulfuron (-methyl), bentazon, benzfendizone, benzobicyclon, benzofenap, benzoylprop (-ethyl), bialaphos, bifenox, bispyribac (-sodium), bromobutide, bromofenoxim, bromoxynil, butachlor, butafenacil (-allyl), butroxydim, butylate, cafenstrole, caloxydim, carbetamide, carfentrazone (-ethyl), chlomethoxyfen, chloramben, chloridazon, chlorimuron (-ethyl), chlornitrofen, chlorsulfuron, chlortoluron, cinidon (-ethyl), cinmethylin, cinosulfuron, clefoxydim, clethodim, clodinafop (-propargyl), clomazone, clomeprop, clopyralid, clopyrasulfuron (-methyl), cloransulam (-methyl), cumyluron, cyanazine, cybutryne, cycloate, cyclosulfamuron, cycloxydim, cyhalofop (-butyl), 2,4-D, 2,4-DB, desmedipham, diallate, dicamba, dichlorprop (—P), diclofop (-methyl), diclosulam, diethatyl (-ethyl), difenzoquat, diflufenican, diflufenzopyr, dimefuron, dimepiperate, dimethachlor, dimethametryn, dimethenamid, dimexyflam, dinitramine, diphenamid, diquat, dithiopyr, diuron, dymron, epropodan, EPTC, esprocarb, ethalfluralin, ethametsulfuron (-methyl), ethofumesate, ethoxyfen, ethoxysulfuron, etobenzanid, fenoxaprop (—P-ethyl), fentrazamide, flamprop (-isopropyl, -isopropyl-L, -methyl), flazasulfuron, florasulam, fluazifop (—P-butyl), fluazolate, flucarbazone (-sodium), flufenacet, flufenpyr, flumetsulam, flumiclorac (-pentyl), flumioxazin, flumipropyn, flumetsulam, fluometuron, fluorochloridone, fluoroglycofen (-ethyl), flupoxam, flupropacil, flurpyrsulfuron (-methyl, -sodium), flurenol (-butyl), fluridone, fluoroxypyr (-butoxypropyl, -meptyl), flurprimidol, flurtamone, fluthiacet (-methyl), fluthiamide, fomesafen, foramsulfuron, glufosinate (-ammonium), glyphosate (-isopropylammonium), halosafen, haloxyfop (-ethoxyethyl, —P-methyl), hexazinone, imazamethabenz (-methyl), imazamethapyr, imazamox, imazapic, imazapyr, imazaquin, imazethapyr, imazosulfuron, iodosulfuron (-methyl, -sodium), ioxynil, isopropalin, isoproturon, isouron, isoxaben, isoxachlortole, isoxaflutole, isoxapyrifop, ketospiradox, lactofen, lenacil, linuron, MCPA, mecoprop, mefenacet, mesotrione, metamitron, metazachlor, methabenzthiazuron, metobenzuron, metobromuron, (alpha-) metolachlor, metosulam, metoxuron, metribuzin, metsulfuron (-methyl), molinate, monolinuron, naproanilide, napropamide, neburon, nicosulfuron, norflurazon, orbencarb, oryzalin, oxadiargyl, oxadiazon, oxasulfuron, oxaziclomefone, oxyfluorfen, paraquat, pelargonic acid, pendimethalin, pendralin, penoxysulam, pentoxazone, pethoxamid, phenmedipham, picolinafen, piperophos, pretilachlor, primisulfuron (-methyl), profluazol, profoxydim, prometryn, propachlor, propanil, propaquizafop, propisochlor, propoxycarbazone (-sodium), propyzamide, prosulfocarb, prosulfuron, pyraflufen (-ethyl), pyrazogyl, pyrazolate, pyrazosulfuron (-ethyl), pyrazoxyfen, pyribenzoxim, pyributicarb, pyridate, pyridatol, pyriftalid, pyriminobac (-methyl), pyrithiobac (-sodium), quinchlorac, quinmerac, quinoclamine, quizalofop (—P-ethyl, —P-tefuryl), rimsulfuron, sethoxydim, simazine, simetryn, sulcotrione, sulfentrazone, sulfometuron (-methyl), sulfosate, sulfosulfuron, tebutam, tebuthiuron, tepraloxydim, terbuthylazine, terbutryn, thenylchlor, thiafluamide, thiazopyr, thidiazimin, thifensulfuron (-methyl), thiobencarb, tiocarbazil, tralkoxydim, triallate, triasulfuron, tribenuron (-methyl), triclopyr, tridiphane, trifluralin, trifloxysulfuron, triflusulfuron (-methyl), tritosulfuron.

Furthermore suitable for the mixtures are known safeners, for example AD-67, BAS-145138, benoxacor, cloquintocet (-mexyl), cyometrinil, 2,4-D, DKA-24, dichlormid, dymron, fenclorim, fenchlorazol (-ethyl), flurazole, fluxofenim, furilazole, isoxadifen (-ethyl), MCPA, mecoprop (—P), mefenpyr (-diethyl), MG-191, oxabetrinil, PPG-1292, R-29148.

A mixture with other known active compounds, such as fungicides, insecticides, acaricides, nematicides, bird repellents, plant nutrients and soil improvers is also possible.

The active compounds can be used as such, in the form of their formulations or in the use forms prepared therefrom by further dilution, such as ready-to-use solutions, suspensions, emulsions, powders, pastes and granules. The application is carried out in a customary manner, for example by watering, spraying, atomizing, broadcasting.

The active compounds according to the invention can be applied both before and after emergence of the plants. They can also be incorporated into the soil prior to sowing.

The application rate of active compound can vary within a relatively large range. It depends essentially on the nature of the desired effect. In general, the application rates are between 1 g and 10 kg of active compound per hectare of soil, preferably between 5 g and 5 kg per ha.

As already mentioned above, it is possible to treat all plants and their parts according to the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering, if appropriate in combination with conventional methods (Genetically Modified Organisms), and parts thereof are treated. The term “parts” or “parts of plants” or “plant parts” has been explained above.

Particularly preferably, plants of the plant cultivars which are in each case commercially available or in use are treated according to the invention. Plant cultivars are understood as meaning plants with specific properties (“traits”) which have been obtained by conventional cultivation, by mutagenesis or else by recombinant DNA techniques. These may be cultivars, biotypes or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or widenings of the activity spectrum and/or an increase in the activity of the substances and compositions that can be used according to the invention also in combination with other agrochemical active compounds, better crop plant growth, increased tolerance of the crop plants to high or low temperatures, increased tolerance of the crop plants to drought or to water or soil salt content, increased flowering performance, easier harvesting, accelerated maturation, higher harvest yields, better quality and/or a higher nutritional value of the harvested products, better storage ability and/or processability of the harvested products which exceed the effects which were actually to be expected are possible.

The preferred transgenic plants or plant cultivars (i.e. those obtained by genetic engineering) which are to be treated according to the invention include all plants which, in the genetic modification, received genetic material which imparted particularly advantageous useful properties (“traits”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products.

Further and particularly emphasized examples of such properties are a better defense of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), corn, soya beans, potatoes, cotton, oilseed rape and also fruit plants (with the fruits apples, pears, citrus fruits and grapes), and particular emphasis is given to corn, soya beans, potatoes, cotton and oilseed rape. Traits that are emphasized are in particular increased defense of the plants against insects by toxins formed in the plants, in particular those formed in the plants by the genetic material from Bacillus thuringiensis (for example by the genes CryIA(a), CryIA(b), CryIA(c), CrylIA, CrylIA, CrylIIIB2, Cry9c, Cry2Ab, Cry3Bb and CrylF and also combinations thereof (hereinbelow referred to as “Bt plants”). Traits which are also particularly emphasized are the increased resistance of plants to fungi, bacteria and viruses by systemic acquired resistance (SAR), systemin, phytoalexins, elicitors and resistance genes and the correspondingly expressed proteins and toxins. Traits that are furthermore particularly emphasized are the increased tolerance of the plants to certain herbicidally active compounds, for example imidazolinones, sulfonylureas, glyphosate or phosphinothricin (for example the “PAT” gene). The genes which impart the desired traits in question can also be present in combination with one another in the transgenic plants. Examples of “Bt plants” which may be mentioned are corn varieties, cotton varieties, soya bean varieties and potato varieties which are sold under the trade names YIELD GARD® (for example corn, cotton, soybeans), KnockOut® (for example corn), StarLink® (for example corn), Bollgard® (cotton), Nucotn® (cotton) and NewLeaf® (potato). Examples of herbicide-tolerant plants which may be mentioned are corn varieties, cotton varieties and soybean varieties which are sold under the trade names Roundup Ready® (tolerance to glyphosates, for example corn, cotton, soybean), Liberty Link® (tolerance to phosphinothricin, for example oilseed rape), IMI® (tolerance to imidazolinones) and STS® (tolerance to sulfonylureas, for example corn). Herbicide-resistant plants (plants bred in a conventional manner for herbicide tolerance) which may be mentioned include the varieties sold under the name Clearfield® (for example corn). Of course, these statements also apply to plant cultivars having these genetic traits or genetic traits still to be developed, which cultivars will be developed and/or marketed in the future.

The plants listed can be treated according to the invention in a particularly advantageous manner with the compounds according to the invention or the active compound mixtures according to the invention, where, in addition to the effective control of the wheat plants, the abovementioned synergistic effects with the transgenic plants or plant cultivars occur. The preferred ranges stated above for the active compounds or mixtures also apply to the treatment of these plants. Particular emphasis is given to the treatment of plants with the compounds or mixtures specifically mentioned in the present text.

The preparation and the use of the active compounds according to the invention is illustrated by the examples below.

### PREPARATION EXAMPLES

### Example 1

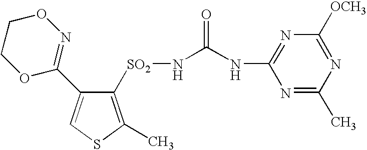

At room temperature (about 20° C.), 0.50 g (1.91 mmol) of 4-(5,6-dihydro-[1,4,2]-dioxazin-3-yl)-2-methylthienyl-3-sulfonamide and 0.29 g (1.91 mmol) of DBU are added successively to a solution of 0.41 g (1.59 mmol) of 2-phenoxycarbonylamino-4-methoxy-6-methyl-1,3,5-triazine in 40 ml of acetonitrile. After 12 hours of stirring at room temperature, the solvent is removed under water pump vacuum, 100 ml of methylene chloride are added, the mixture is washed with 2N hydrochloric acid and with water and dried over sodium sulfate and the added solvent is finally removed under water pump vacuum. The residue is recrystallized from isopropanol, filtered off and dried.

This gives 0.40 g (59% of theory) of N-(4-methoxy-6-methyl-1,3,5-triazin-2-yl)-N′-(4-(5,6-dihydro-[1,4,2]-dioxazin-3-yl)-2-methylthien-3-ylsu lfonyl)urea of melting point 200° C.

Analogously to example 1 and in accordance with the general description of the preparation process according to the invention, it is also possible to prepare, for example, the compounds of the formula (I) listed in table 1 below.

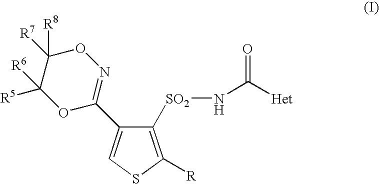

TABLE 1
Examples of compounds of the formula (I)

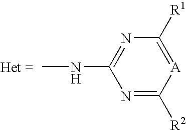

| Ex. | | | | | | Melting point |
|---|---|---|---|---|---|---|
| No. | A | R¹ | R² | R | R⁵, R⁶, R⁷, R⁸ | (° C.) |
| 2 | CH | OCH₃ | OCH₃ | CH₃ | H | 211 |
| 3 | CH | OCH₃ | Cl | CH₃ | H | 97 |
| 4 | N | OCH₃ | OCH₃ | CH₃ | H | 199 |

### Example 5

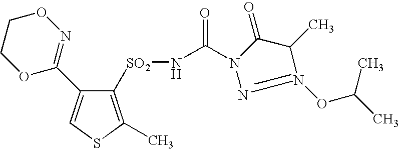

At room temperature (about 20° C.), 0.50 g (1.91 mmol) of 4-(5,6-dihydro-[1,4,2]-dioxazin-3-yl)-2-methylthienyl-3-sulfonamide and 0.29 g (1.91 mmol) of DBU are added successively to a solution of 0.44 g (1.59 mmol) of 5-isopropyloxy-4-methyl-2-phenoxycarbonyl-2,4-dihydro-3H-1,2,4-triazol-3-one in 40 ml of acetonitrile. After 12 hours of stirring at room temperature, the solvent is removed under water pump vacuum, 100 ml of methylene chloride are added, the mixture is washed with 2N hydrochloric acid and with water and dried over sodium sulfate and the added solvent is finally removed under water pump vacuum. The residue is recrystallized from isopropanol, filtered off and dried.

This gives 0.5 g (71% of theory) of 5-isopropyloxy-4-methyl-2-[[4-(5,6-dihydro-[1,4,2]-dioxazin-3-yl)-2-methylthien-3-yl]sulfonylaminocarbonyl]-2,4-dihydro-3H-1,2,4-triazol-3-one of melting point 199° C.

Analogously to example 5 and in accordance with the general description of the preparation process according to the invention, it is also possible to prepare, for example, the compounds of the formula (I) listed in table 1 (continued) below.

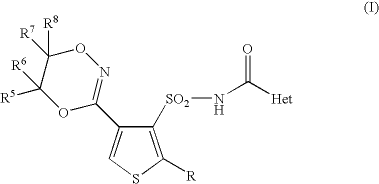

TABLE 1
Examples of compounds of the formula (I)

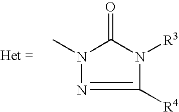

| Ex. | | | | | Melting point |
|---|---|---|---|---|---|
| No. | R³ | R⁴ | R | R⁵, R⁶, R⁷, R⁸ | (° C.) |
| 6 | CH₃ | OCH₃ | CH₃ | H | 157 |
| 7 | CH₃ | OCH₂CH₃ | CH₃ | H | 185 |
| 8 | CH₃ | O-(n-propyl) | CH₃ | H | 177 |
| 9 | 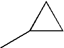 | OCH₃ | CH₃ | H | 204 |
| 10 | 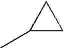 | OCH₂CH₃ | CH₃ | H | 202 |
| 11 | 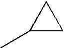 | O-(n-propyl) | CH₃ | H | 190 |
| 12 | 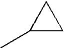 | O-(i-propyl) | CH₃ | H | 200 |

Starting materials of the formula (III):

### Example (III-1)

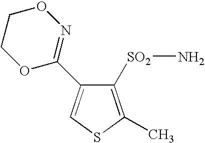

27.8 g (0.4 mol) of hydroxylamine hydrochloride are suspended in 400 ml of methanol, and, at room temperature (about 20° C.), a solution of 44.8 g (0.8 mol) of potassium hydroxide in 400 ml of methanol is added. At room temperature, 47 g (0.7 mol) of methyl 2-methyl-3-sulfamoylthienyl-4-carboxylate are added a little at a time, and the mixture is stirred at 40° C. for 12 hours. After cooling to room temperature, 27.6 g (0.2 mol) of potassium carbonate are added and 185.8 g (0.92 mol) of 1,2-dibromoethane are added dropwise. The mixture is then stirred at 60° C. for another 12 hours. After cooling to room temperature, the solvent is removed under water pump vacuum and the residue that remains is digested with 400 ml of methylene chloride and 200 ml of a 25% strength sodium dihydrogen phosphate solution. The precipitate formed is filtered off and dried.

This gives 11.8 g (23% of theory) of 4-(5,6-dihydro-[1,4,2]-dioxazin-3-yl)-2-methylthienyl-3-sulfonamide of melting point 48° C.

### WORKING EXAMPLES

### Example A

### Post-Emergence Test

Seeds of mono- and dicotyledonous weed and crop plants are placed in sandy loam soil in wood fiber pots, covered with soil and cultivated in a greenhouse under good growth conditions. 2 to 3 weeks after sowing, the trial plants are treated at the one-leaf stage. The test compounds, formulated as wettable powders (WP), are sprayed at various dosages with a water application rate of 800 I/ha (converted) and with addition of 0.2% of welting agent onto the green parts of the plants. After the trial plants have stood in the greenhouse for about 3 weeks under optimum growth conditions, the effect of the products is scored visually in comparison to untreated controls (herbicidal effect in percent (%): 100% effect=plants have died, 0% effect=like control plants). In this test, for example, the compounds of preparation examples 1, 3, 6, 7 and 9 are highly effective against weeds.

TABLE A1
Post-emergence test/greenhouse

| | | | | | | | | |
|---|---|---|---|---|---|---|---|---|
| Active compound of | Application | | | | | | | |
| preparation example No. | rate (g of ai/ha) | Amaranthus | Lolium | Setaria | Sinapis | Stellaria | Cyperus | Echinochloa |

| | | | | | | | | |
|---|---|---|---|---|---|---|---|---|
| (1) | 80 | 90 | 90 | 90 | 90 | 90 | 100 | 90 |
| (3) | 80 | 90 | 90 | 80 | 90 | 90 | 100 | 90 |
| (6) | 80 | 90 | 90 | 80 | 80 | 90 | 80 | 80 |
| (7) | 80 | 90 | 90 | 90 | 80 | 90 | 90 | 90 |
| (9) | 80 | 100 | 90 | 90 | 80 | 90 | 70 | 90 |

1. A compound of the formula (I) 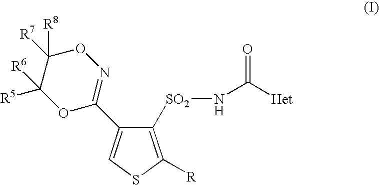
in which
Het is 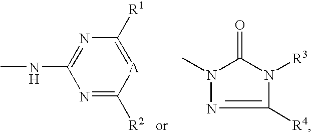
A is nitrogen or a CH grouping,
R is hydrogen, cyano, nitro, halogen, is in each case optionally cyano-, halogen- or C₁-C₄-alkoxy-substituted alkyl, alkoxy, alkoxycarbonyl, alkylthio, alkylsulfinyl or alkylsulfonyl having in each case 1 to 6 carbon atoms in the alkyl group, or is in each case optionally cyano- or halogen-substituted alkenyl, alkynyl, alkenyloxy or alkynyloxy having in each case 2 to 6 carbon atoms in the alkenyl or alkynyl group,
R¹ is hydrogen, is halogen, is in each case optionally cyano-, halogen- or C₁-C₄-alkoxy-substituted alkyl, alkoxy, alkylthio, alkylamino or dialkylamino having in each case 1 to 4 carbon atoms in the alkyl groups, or is in each case optionally cyano-, halogen-, C₁-C₄-alkyl- or C₁-C₄-alkoxy-substituted phenoxy, oxetanyloxy, furyloxy or tetrahydrofuryloxy,
R² is hydrogen, is halogen, is in each case optionally cyano-, halogen- or C₁-C₄-alkoxy-substituted alkyl, alkoxy, alkylthio, alkylamino or dialkylamino having in each case 1 to 4 carbon atoms in the alkyl groups, or is in each case optionally cyano-, halogen-, C₁-C₄-alkyl- or C₁-C₄-alkoxy-substituted phenoxy, oxetanyloxy, furyloxy or tetrahydrofuryloxy,
R³ is hydrogen, hydroxyl, amino, cyano, is C₂-C₁₀-alkylideneamino, is optionally fluorine-, chlorine-, bromine-, cyano-, C₁-C₄-alkoxy-, C₁-C₄-alkyl-carbonyl- or C₁-C₄-alkoxy-carbonyl-substituted alkyl having 1 to 6 carbon atoms, is in each case optionally fluorine-, chlorine- and/or bromine-substituted alkenyl or alkynyl having in each case 2 to 6 carbon atoms, is in each case optionally fluorine-, chlorine-, bromine-, cyano-, C₁-C₄-alkoxy- or C₁-C₄-alkoxy-carbonyl-substituted alkoxy, alkylamino or alkylcarbonylamino having in each case 1 to 6 carbon atoms in the alkyl group, is alkenyloxy having 3 to 6 carbon atoms, is dialkylamino having in each case 1 to 4 carbon atoms in the alkyl groups, is in each case optionally fluorine-, chlorine-, bromine-, cyano- and/or C₁-C₄-alkyl-substituted cycloalkyl, cycloalkylamino or cycloalkylalkyl having in each case 3 to 6 carbon atoms in the alkyl group and, if appropriate, 1 to 4 carbon atoms in the alkyl moiety, or is in each case optionally fluorine-, chlorine-, bromine-, cyano-, nitro-, C₁-C₄-alkyl-, trifluoromethyl- and/or C₁-C₄-alkoxy-substituted aryl or arylalkyl having in each case 6 or 10 carbon atoms in the aryl group and, if appropriate, 1 to 4 carbon atoms in the alkyl moiety.
R⁴ is hydrogen, hydroxyl, mercapto, amino, cyano, fluorine, chlorine, bromine, iodine, is optionally fluorine-, chlorine-, bromine-, cyano-, C₁-C₄-alkoxy-, C₁-C₄-alkyl-carbonyl- or C₁-C₄-alkoxy-carbonyl-substituted alkyl having 1 to 6 carbon atoms, is in each case optionally fluorine-, chlorine- and/or bromine-substituted alkenyl or alkynyl having in each case 2 to 6 carbon atoms, is in each case optionally fluorine-, chlorine-, cyano-, C₁-C₄-alkoxy- or C₁-C₄-alkoxy-carbonyl-substituted alkoxy, alkylthio, alkylamino or alkylcarbonylamino having in each case 1 to 6 carbon atoms in the alkyl group, is alkenyloxy, alkynyloxy, alkenylthio, alkynylthio, alkenylamino or alkynylamino having in each case 3 to 6 carbon atoms in the alkenyl or alkynyl group, is dialkylamino having in each case 1 to 4 carbon atoms in the alkyl groups, is in each case optionally fluorine-, chlorine-, bromine-, cyano- and/or C₁-C₄-alkyl-substituted cycloalkyl, cycloalkenyl, cycloalkyloxy, cycloalkylthio, cycloalkylamino, cycloalkylalkyl, cycloalkylalkoxy, cycloalkylalkylthio or cycloalkylalkylamino having in each case 3 to 6 carbon atoms in the cycloalkyl or cycloalkenyl group and, if appropriate, 1 to 4 carbon atoms in the alkyl moiety, or is in each case optionally fluorine-, chlorine-, bromine-, cyano-, nitro-, C₁-C₄-alkyl-, trifluoromethyl-, C₁-C₄-alkoxy- and/or C₁-C₄-alkoxy-carbonyl-substituted aryl, arylalkyl, aryloxy, arylalkoxy, arylthio, arylalkylthio, arylamino or arylalkylamino having in each case 6 or 10 carbon atoms in the aryl group and, if appropriate, 1 to 4 carbon atoms in the alkyl moiety, and
R⁵, R⁶, R⁷ and R⁸ independently of one another are hydrogen, halogen, cyano or thiocyanato or are in each case optionally halogen-substituted alkyl, alkoxy, alkylthio, alkylsulfinyl, alkylsulfonyl, alkylamino, alkylcarbonyl, alkoxycarbonyl or alkylaminocarbonyl having in each case 1 to 3 carbon atoms in the alkyl moiety,
or a salt of a compound of the formula (I).

2. The compound as claimed in claim 1, wherein
R is hydrogen, cyano, fluorine, chlorine, bromine, is in each case optionally cyano-, fluorine-, chlorine-, methoxy- or ethoxy-substituted methyl, ethyl, n- or i-propyl, n-, i-, s- or t-butyl, methoxy, ethoxy, n- or i-propoxy, methoxycarbonyl, ethoxycarbonyl, n- or i-propoxycarbonyl, methylthio, ethylthio, n- or i-propylthio, methylsulfinyl, ethylsulfinyl, methylsulfonyl or ethylsulfonyl, or is in each case optionally cyano-, fluorine- or chlorine-substituted propenyl, butenyl, propynyl, butynyl, propenyloxy, butenyloxy, propynyloxy or butynyloxy,
R¹ is fluorine, chlorine, bromine, iodine, or is in each case optionally cyano-, fluorine-, chlorine-, methoxy- or ethoxy-substituted methyl, ethyl, n- or i-propyl, methoxy, ethoxy, n- or i-propoxy, methylthio, ethylthio, n- or i-propylthio, methylamino, ethylamino, n- or i-propylamino, dimethylamino or diethylamino,
R² is fluorine, chlorine, bromine, iodine, or is in each case optionally cyano-, fluorine-, chlorine-, methoxy- or ethoxy-substituted methyl, ethyl, n- or i-propyl, methoxy, ethoxy, n- or i-propoxy, methylthio, ethylthio, n- or i-propylthio, methylamino, ethylamino, n- or i-propylamino, dimethylamino or diethylamino,
R³ is hydrogen, hydroxyl, amino, is in each case optionally fluorine-, chlorine-, cyano-, methoxy- or ethoxy-substituted methyl, ethyl, n- or i-propyl, n-, i-, s- or t-butyl, is in each case optionally fluorine-, chlorine- and/or bromine-substituted ethenyl, propenyl, butenyl, propynyl or butynyl, is in each case optionally fluorine-, chlorine-, cyano-, methoxy- or ethoxy-substituted methoxy, ethoxy, n- or i-propoxy, n-, i-, s- or t-butoxy, methylamino, ethylamino, n- or i-propylamino, n-, i-, s- or t-butylamino, is propenyloxy or butenyloxy, is dimethylamino or diethylamino, is in each case optionally fluorine-, chlorine-, methyl- and/or ethyl-substituted cyclopropyl, cyclobutyl, cyclopentyl, cyclohexyl, cyclopropylamino, cyclobutylamino, cyclopentylamino, cyclohexylamino, cyclopropylmethyl, cyclobutylmethyl, cyclopentylmethyl or cyclohexylmethyl, or is in each case optionally fluorine-, chlorine-, methyl-, trifluoromethyl- and/or methoxy-substituted phenyl or benzyl,
R⁴ is hydrogen, hydroxyl, mercapto, amino, cyano, fluorine, chlorine, bromine, is in each case optionally fluorine-, chlorine-, cyano-, methoxy-, ethoxy-, n- or i-propoxy-, acetyl-, propionyl-, n- or i-butyroyl-, methoxycarbonyl-, ethoxycarbonyl-, n- or i-propoxycarbonyl-substituted methyl, ethyl, n- or i-propyl, n-, i-, s- or t-butyl, is in each case optionally fluorine-, chlorine- and/or bromine-substituted ethenyl, propenyl, butenyl, ethynyl, propynyl or butynyl, is in each case optionally fluorine-, chlorine-, cyano-, methoxy-, ethoxy-, n- or i-propoxy-, methoxycarbonyl-, ethoxycarbonyl-, n- or i-propoxycarbonyl-substituted methoxy, ethoxy, n- or i-propoxy, n-, i-, s- or t-butoxy, methylthio, ethylthio, n- or i-propylthio, n-, i-, s- or t-butylthio, methylamino, ethylamino, n- or i-propylamino, n-, i-, s- or t-butylamino, acetylamino or propionylamino, is propenyloxy, butenyloxy, ethynyloxy, propynyloxy, butynyloxy, propenylthio, butenylthio, propynylthio, butynylthio, propenylamino, butenylamino, propynylamino or butynylamino, is dimethylamino, diethylamino or dipropylamino, is in each case optionally fluorine-, chlorine-, methyl- and/or ethyl-substituted cyclopropyl, cyclobutyl, cyclopentyl, cyclohexyl, cyclopentenyl, cyclohexenyl, cyclopropyloxy, cyclobutyloxy, cyclopentyloxy, cyclohexyloxy, cyclopropylthio, cyclobutylthio, cyclopentylthio, cyclohexylthio, cyclopropylamino, cyclobutylamino, cyclopentylamino, cyclohexylamino, cyclopropylmethyl, cyclobutylmethyl, cyclopentyl-methyl, cyclohexylmethyl, cyclopropylmethoxy, cyclobutylmethoxy, cyclopentylmethoxy, cyclohexylmethoxy, cyclopropylmethylthio, cyclo-butylmethylthio, cyclopentylmethylthio, cyclohexylmethylthio, cyclo-propylmethylamino, cyclobutylmethylamino, cyclopentylmethylamino or cyclohexylmethylamino, or is in each case optionally fluorine-, chlorine-, bromine-, methyl-, trifluoromethyl-, methoxy- or methoxy-carbonyl-substituted phenyl, benzyl, phenoxy, benzyloxy, phenylthio, benzylthio, phenylamino or benzylamino, and R⁵, R⁶, R⁷ and R⁸ independently of one another are hydrogen or methyl,
or a sodium, potassium, magnesium, calcium, ammonium-, C₁-C₄-alkyl-ammonium, di-(C₁-C₄-alkyl)-ammonium, tri-(C₁-C₄-alkyl)-ammonium, tetra-(C₁-C₄-alkyl)-ammonium, tri-(C₁-C₄-alkyl)-sulfonium, C₅- or C₆₋cycloalkyl-ammonium or di-(C₁-C₂-alkyl)-benzylammonium salt of this compound.

3. The compound as claimed in claim 1, wherein
R is fluorine, chlorine, bromine, is in each case optionally cyano-, fluorine-, chlorine-, methoxy- or ethoxy-substituted methyl, ethyl, n- or i-propyl, n-, i-, s- or t-butyl, methoxy, ethoxy, n- or i-propoxy,
R¹ is fluorine, chlorine, bromine, or iodine, or is optionally fluorine- or chlorine-substituted methyl, ethyl, n- or i-propyl, methoxy, ethoxy, n- or i-propoxy or is dimethylamino,
R² is fluorine, chlorine, bromine, or iodine, or is optionally fluorine- or chlorine-substituted methyl, ethyl, n- or i-propyl, methoxy, ethoxy, n- or i-propoxy or is dimethylamino,
R³ is methyl, ethyl, n- or i-propyl or cyclopropyl,
R⁴ is methoxy, ethoxy, n- or i-propoxy, n-, i-, s- or t-butoxy, and
R⁵, R⁶, R⁷ and R⁸ are hydrogen,
or a sodium, potassium, magnesium, calcium, ammonium-, C₁-C₄-alkyl-ammonium, di-(C₁-C₄-alkyl)-ammonium, tri-(C₁-C₄-alkyl)-ammonium, tetra-(C₁-C₄-alkyl)-ammonium, tri-(C₁-C₄-alkyl)-sulfonium, C₅- or C₆-cycloalkyl-ammonium or di-(C₁-C₂-alkyl)-benzylammonium salt of a compound of the formula (I).

4. A process for preparing compounds as claimed in claim 1, which comprises
(a) reacting substituted aminoazines of the formula (II) 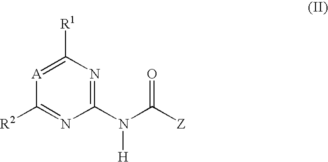
in which
A, R¹ and R² are as defined in claim 1 and
Z is halogen, alkoxy or aryloxy
with thiophene derivatives of the formula (III) 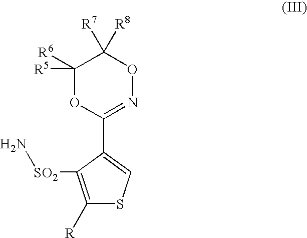
in which
R and R⁵ to R⁸ are as defined in claim 1,
if appropriate in the presence of a reaction auxiliary and if appropriate in the presence of a diluent,
or (b) reacting substituted thiophene-3-sulfonamides of the formula (III) 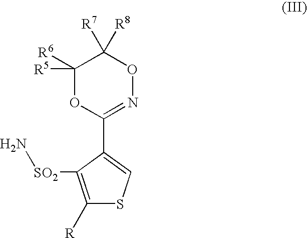
in which R and R⁵ to R⁸ are as defined in claim 1
with substituted triazolinones of the formula (IV) 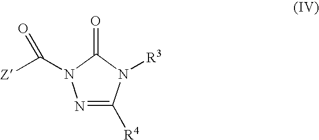
in which
R³ and R⁴ are as defined in claim 1 and
Z′ is halogen, alkoxy, aryloxy or arylalkoxy,
if appropriate in the presence of a reaction auxiliary and if appropriate in the presence of a diluent,
and, if appropriate, converting the compounds of the formula (I) obtained by process (a) or (b) by customary methods into salts.

5. A method for controlling unwanted vegetation, which comprises allowing at least one compound as claimed in any of claims 1 to 3 to act on unwanted plants, parts of plants and/or their habitat.

6. The use of at least one compound as claimed in any of claims 1 to 3 for controlling unwanted plants.

7. A herbicidal composition, which comprises one or more compounds as claimed in any of claims 1 to 3 and customary extenders and/or surfactants.

8. A herbicidal composition, which comprises one or more compounds as claimed in any of claims 1 to 3 and one or more other agrochemically active compounds.

9. A compound of the formula (III) 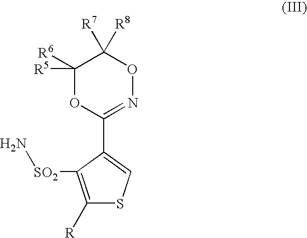
in which
R and R⁵ to R⁸ are as defined in claim 1.

10. The use of compounds of the formula (III) for preparing compounds as claimed in any of claims 1 to 3.
